(19) United States
(12) Patent Application Publication
(10) Pub. No.: US 2022/0395650 A1
(43) Pub. Date: Dec. 15, 2022

(54) SYSTEM AND METHOD OF MANUFACTURE FOR INHALATION DEVICE

(71) Applicant: MYLAN PHARMA UK LIMITED, Hertfordshire (GB)

(72) Inventors: Matthew Danson Parker, Kent (GB); Michael John Salter, Kent (GB)

(73) Assignee: MYLAN PHARMA UK LIMITED, Hertfordshire (GB)

(21) Appl. No.: 17/621,610

(22) PCT Filed: Jun. 24, 2020
(86) PCT No.: PCT/IB2020/055991

§ 371 Date: Dec. 21, 2021

Related U.S. Application Data
(60) Provisional application No. 62/866,163, filed on Jun. 25, 2019.

(51) Int. Cl. A61M 15/00; B65B 9/04

(52) U.S. Cl. CPC A61M 15/0048; B65B 9/04; A61M 2202/064

(57) ABSTRACT

A method for fabricating an inhalation device includes the steps of disposing a medicament carrier with a first member, the carrier being connected with a layer; engaging a second member with the first member to support the layer therebetween, the second member including a cutting surface and defining a cavity; and translating the carrier relative to the members to dispose the carrier with the cavity and cut the layer about the carrier. In some embodiments, manufacturing systems, manufacturing apparatus, inhalation devices, dispensing devices, and medicaments are disclosed.

### CROSS REFERENCE TO RELATED APPLICATION(S)

The present application claims priority to U.S. Provisional Patent Application No. 62/866,163, filed on Jun. 25, 2019, which is incorporated herein by reference in its entirety.

### BACKGROUND

### Field

The present invention generally relates to inhalation technology for drug delivery and more particularly to a method for making a drug delivery device.

### Description of Related Art

Inhalation technology for drug delivery has been employed for treating various lung, intrapulmonary and extrapulmonary diseases such as asthma, chronic obstructive pulmonary disease, cystic fibrosis, and other pulmonary infections. Various devices are known, for example, nebulizers, pressurized metered-dose inhalers, and dry powder inhalers (DPIs) for dispensing medicament in connection with treatment of several lung diseases and pathological conditions.

DPIs provide direct inhalation of a powdered drug from which individual doses can be metered. The DPIs typically include a carrier that delivers the doses from a bulk reservoir, capsule, or blister package, for inhalation by a patient. In an effort to avoid caking or contamination of the doses, individual dose containers can be provided with the carrier. During manufacture of a DPI, an aluminum backing can be applied to a surface of the carrier for containment of the doses with the carrier.

### SUMMARY

According to an example of the present disclosure, an improvement over prior DPI manufacturing technologies is provided.

According to a particular example of the present disclosure, a method for fabricating an inhalation device is provided. The method comprises the steps of: disposing a medicament carrier within a first member, the carrier being connected with a layer; engaging a second member with the first member to support the layer therebetween, the second member including a cutting surface and defining a cavity; and translating the carrier relative to the members to dispose the carrier within the cavity and cut the layer about the carrier.

The first member may include a guide, and the step of disposing may include aligning the carrier with the guide.

The cutting surface may have a blunt edge.

The step of translating may include engaging the carrier such that the carrier translates along an axis of the members to cut the layer about the carrier. The step of translating may include engaging the carrier with a fluid.

The method may further comprise the step of expelling the carrier from the cavity with a release actuator.

According to a particular example of the present disclosure, a method for fabricating an inhalation device is provided. The method comprises the steps of: disposing a medicament dose carrier within a base plate, the base plate including an inner surface and a central guide that define a base cavity, the carrier including a disc being positioned onto the guide and connected with a foil web; engaging a lid plate including an inner surface and a central guide that define a lid cavity with the base plate to support the foil web therebetween, the lid plate further including a cutting surface having a blunt edge; engaging the carrier with an actuator such that the disc translates along the guide of the lid plate to cut the foil web about a perimeter of the disc; and expelling the carrier from the lid cavity with a release actuator.

According to a particular example of the present disclosure, a manufacturing apparatus is provided. The manufacturing apparatus comprises a first plate that defines an axis and is configured for disposal of a medicament carrier; a second plate being aligned and engageable with the first plate to support a layer therebetween, the carrier being connected to the layer and at least one of the plates including a cutting surface; and an actuator being engageable with the carrier such that the carrier translates along the axis and cuts the layer about the carrier.

The cutting surface may have a blunt edge.

Each of the plates may include an inner surface and a central guide that define a cavity.

At least one of the plates may include a central guide engageable with the carrier.

The first plate may include at least one opening for disposal of the actuator.

The actuator may include a fluid.

The actuator may include a pin.

The first plate may include at least one opening for disposal of the actuator, and the second plate may include at least one opening for disposal of a release actuator that expels the carrier from the second plate.

The layer may include a foil web.

The layer may include a membrane cut to a profile of the carrier.

The carrier may include a disc having a plurality of spaced-apart pockets.

The layer may be sealed to the carrier.

The carrier may include a disc having a plurality of spaced-apart dose pockets.

The manufacturing apparatus may further comprise a foil web sealed to a first planar surface of the carrier and the layer being sealed to a second planar surface of the carrier.

Further preferred and non-limiting embodiment or aspects will now be described in the following numbered clauses.

Clause 1: A method for fabricating an inhalation device, the method comprising the steps of: disposing a medicament carrier within a first member, the carrier being connected with a layer; engaging a second member with the first member to support the layer therebetween, the second member including a cutting surface and defining a cavity; and translating the carrier relative to the members to dispose the carrier within the cavity and cut the layer about the carrier.

Clause 2: The method according to clause 1, wherein the first member includes a guide and the step of disposing includes aligning the carrier with the guide.

Clause 3: The method according to clause 1 or clause 2, wherein the cutting surface has a blunt edge.

Clause 4: The method according to any one of clauses 1-3, wherein the step of translating includes engaging the carrier such that the carrier translates along an axis of the members to cut the layer about the carrier.

Clause 5: The method according to any one of clauses 1-4, wherein the step of translating includes engaging the carrier with a fluid.

Clause 6: The method according to any one of clauses 1-5, further comprising the step of expelling the carrier from the cavity with a release actuator.

Clause 7: A method for fabricating an inhalation device, the method comprising the steps of: disposing a medicament dose carrier within a base plate, the base plate including an inner surface and a central guide that define a base cavity, the carrier including a disc being positioned onto the guide and connected with a foil web; engaging a lid plate including an inner surface and a central guide that define a lid cavity with the base plate to support the foil web therebetween, the lid plate further including a cutting surface having a blunt edge; engaging the carrier with an actuator such that the disc translates along the guide of the lid plate to cut the foil web about a perimeter of the disc; and expelling the carrier from the lid cavity with a release actuator.

Clause 8: A manufacturing apparatus comprising: a first plate that defines an axis and is configured for disposal of a medicament carrier; a second plate being aligned and engageable with the first plate to support a layer therebetween, the carrier being connected to the layer and at least one of the plates including a cutting surface; and an actuator being engageable with the carrier such that the carrier translates along the axis and cuts the layer about the carrier.

Clause 9: The manufacturing apparatus according to clause 8, wherein the cutting surface has a blunt edge.

Clause 10: The manufacturing apparatus according to clause 8 or clause 9, wherein each of the plates include an inner surface and a central guide that define a cavity.

Clause 11: The manufacturing apparatus according to any one of clauses 8-10, wherein at least one of the plates includes a central guide engageable with the carrier.

Clause 12: The manufacturing apparatus according to any one of clauses 8-11, wherein the first plate includes at least one opening for disposal of the actuator.

Clause 13: The manufacturing apparatus according to any one of clauses 8-12, wherein the actuator includes a fluid.

Clause 14: The manufacturing apparatus according to any one of clauses 8-12, wherein the actuator includes a pin.

Clause 15: The manufacturing apparatus according to any one of clauses 8-14, wherein the first plate includes at least one opening for disposal of the actuator and the second plate includes at least one opening for disposal of a release actuator that expels the carrier from the second plate.

Clause 16: The manufacturing apparatus according to any one of clauses 8-15, wherein the layer includes a foil web.

Clause 17: The manufacturing apparatus according to any one of clauses 8-16, wherein the layer includes a membrane cut to a profile of the carrier.

Clause 18: The manufacturing apparatus according to any one of clauses 8-17, wherein the carrier includes a disc having a plurality of spaced-apart pockets.

Clause 19: The manufacturing apparatus according to any one of clauses 8-18, wherein the layer is sealed to the carrier.

Clause 20: The manufacturing apparatus according to any one of clauses 8-19, wherein the carrier includes a disc having a plurality of spaced-apart dose pockets.

Clause 21: The manufacturing apparatus according to any one of clauses 8-20, further comprising a foil web sealed to a first planar surface of the carrier and the layer being sealed to a second planar surface of the carrier.

These and other features and characteristics of the present invention, as well as the methods of operation and functions of the related elements of structures and the combination of parts and economies of manufacture, will become more apparent upon consideration of the following description and with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and the claims, the singular forms of “a”, “an”, and “the” include plural referents unless the context clearly dictates otherwise.

### BRIEF DESCRIPTION OF THE DRAWINGS

FIG. 1 is a perspective view of components of one embodiment of an inhalation device in accordance with the principles of the present disclosure;

FIG. 2 is a side view of the inhalation device shown in FIG. 1;

FIG. 3 is a perspective view, with parts separated, of components of one embodiment of an inhalation device in accordance with the principles of the present disclosure;

FIG. 4 is a flow diagram illustrating representative steps of employing one embodiment of a manufacturing system in accordance with the principles of the present disclosure;

FIG. 5 is a schematic arrangement of components of one embodiment of a manufacturing system in accordance with the principles of the present disclosure;

FIG. 6 is a top view of components of one embodiment of a manufacturing system in accordance with the principles of the present disclosure;

FIGS. 7A-7D are top views of components of one embodiment of a system in accordance with the principles of the present disclosure;

FIGS. 8A-8C are schematic side views of components of one embodiment of a system in accordance with the principles of the present disclosure;

FIG. 9 is a side view of components of one embodiment of a manufacturing system in accordance with the principles of the present disclosure; and

FIG. 10 is a cross sectional view of the filling machine shown in FIG. 5 according to one embodiment of the present disclosure.

### DETAILED DESCRIPTION

For purposes of the description hereinafter, the terms “end”, “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations and step sequences, except where expressly specified to the contrary. It is also to be understood that the specific devices and processes illustrated in the attached drawings and described in the following specification are simply exemplary embodiments or aspects of the invention. Hence, specific dimensions and other physical characteristics related to the embodiments or aspects disclosed herein are not to be considered as limiting. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

The exemplary embodiments of drug delivery devices employing inhalation technology are discussed in terms of dispensing devices for the treatment of various diseases, illness and/or ailments, and more particularly, in terms of related methods of manufacturing, manufacturing systems, and apparatus for making such drug delivery devices. In some embodiments, a medicament packaging system is provided that includes an inhalation device for direct inhalation of a powdered drug. In some embodiments, a manufacturing system is provided for making the inhalation device. In some embodiments, the present manufacturing system includes a press and die apparatus for cutting a layer that supports medication with the inhalation device. In some embodiments, the present manufacturing system is employed with a method that cuts a layer supporting medication with the inhalation device.

In some embodiments, the present system includes an inhalation device having a medication dose carrier, such as, for example, a cartridge disc. In some embodiments, the cartridge disc includes a flexible sheet, such as, for example, a foil disposed with a top surface and a bottom surface of the disc. In some embodiments, the cartridge disc includes a dose carrier with pockets that may include one or more layers of medicament, such as, for example, a powdered inhalant. In some embodiments, the cartridge disc comprises a die portion of a manufacturing apparatus of a manufacturing system.

In some embodiments, the present manufacturing system is employed with a method including the step of cutting and/or attaching a foil with the disc. In some embodiments, the present manufacturing system includes a manufacturing apparatus having a member, such as, for example, a base and a member, such as, for example, a lid.

In some embodiments, the present manufacturing system is employed with a method including the step of sealing a flexible sheet, such as, for example, a foil web to a cartridge disc. In some embodiments, the method includes the step of engaging the cartridge disc with a manufacturing apparatus having a cutting device. In some embodiments, a shape of a cartridge disc is directly pressed out of a foil web about a profile of the disc. In some embodiments, the disc includes a cutting surface comprising a blunt edge to facilitate cutting of the foil about a profile of the disc. In some embodiments, the disc includes a cutting surface comprising a 0.4 millimeter (mm), 45 angular degree chamfered edge to facilitate cutting a foil web about a profile of the disc. In some embodiments, the foil web is sheared around an edge of the disc. In some embodiments, the method includes the step of firmly holding the foil web circumferentially around an edge of the disc and cutting the foil about a profile of the disc.

In some embodiments, the present system includes an inhalation device having a cartridge disc with both top and bottom lidding foils. In some embodiments, the disc is sealed on a bottom surface to resist and/or prevent receptacles filled with medicament from being dislodged from the disc when a top foil is applied to a top surface of the disc.

In some embodiments, the present manufacturing system is employed with a method including the step of sealing a cartridge disc to a foil web. In some embodiments, the method includes the step of placing the disc into a manufacturing apparatus. In some embodiments, the method includes the step of clamping the disc with a manufacturing apparatus and the foil to keep the foil taut with the manufacturing apparatus. In some embodiments, the method includes the step of applying a force of compressed air to a surface of the disc to translate the disc for cutting the foil with a cutting device of the manufacturing apparatus. In some embodiments, the method includes the step of forcing the disc into a female portion of the cutting device. In some embodiments, the method includes the step of shearing the foil around the periphery of the disc cleanly, taking the shape of the disc.

In some embodiments, the present system includes an inhalation device having a cartridge disc with a lid that is a mirror image of the base. In some embodiments, the base includes at least one notch and the lid includes at least one pin to facilitate relative alignment and/or assembly. In some embodiments, the base and the lid include a tolerance relative to the dimensions of the disc. In some embodiments, the tolerance may be +/−0.1 mm.

In some embodiments, the present manufacturing system is employed with a method including the step of placing a cartridge disc and a foil web onto a base. In some embodiments, the method includes the step of locating a notch on the base. In some embodiments, the method includes the step of aligning a lid with the base and clamping the lid and the base together. In some embodiments, the method includes the step of pushing and/or blowing the disc into a cavity disposed in the lid. In some embodiments, translation into the lid provides a clear and clean cut of the foil around the disc. In some embodiments, the method includes the step of removing the lid. In some embodiments, the method includes the step of removing the disc from the lid by pushing the lid out with a push-rod.

As used in the specification and including the appended claims, “treating” or “treatment” of a disease or condition refers to performing a procedure that may include administering one or more drugs to a patient (human, normal or otherwise, or other mammal) in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation and/or alleviating pain.

The following discussion includes a description of methods of manufacturing, manufacturing systems and apparatus for making drug delivery devices in accordance with the principles of the present disclosure, and in particular, such devices employing inhalation technology for the treatment of various diseases, illness and/or ailments. Alternate embodiments are disclosed. Reference is made in detail to the exemplary embodiments of the present disclosure, which are illustrated in the accompanying figures. Turning to FIGS. 1-10, there are illustrated components of a manufacturing system 10 employed for fabrication of one or more components of an inhalation device 12.

The components of manufacturing system 10, individually or collectively, can be fabricated from materials suitable for preparation, treatment, molding, cutting, production and/or manufacture of one or more components of a drug delivery device. The components of inhalation device 12, individually or collectively, can be fabricated from materials suitable for storage and dispensing of medication. In some embodiments, such materials include metals, ceramics, synthetic polymers such as thermoplastics, semi-rigid and rigid materials, elastomers, fabric and/or their composites. Various components of manufacturing system 10 and/or inhalation device 12 may have material composites to achieve various desired characteristics such as strength, rigidity, elasticity, compliance, biomechanical performance, and/or durability. The components of the manufacturing system 10 and/or the inhalation device 12 may be monolithically formed or integrally connected or may include fastening elements and/or instruments, as described herein.

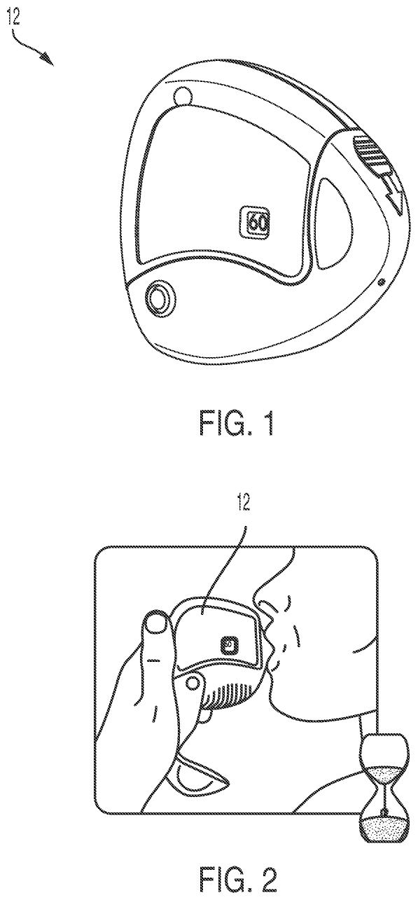

The inhalation device 12 includes a dry powder inhaler (DPI) device and may be utilized for delivery of a medicament by oral and/or nasal inhalation. The inhalation device 12 includes a medicament carrier 14, as described herein. In some embodiments, the medicament carrier 14 is compatible as a DPI device, as shown in FIGS. 1 and 2. See, for example, the DPI systems and devices disclosed in U.S. Pat. No. 9,399,103, the entirety of which is hereby incorporated by reference herein.

The medicament carrier 14 includes multiple individually packed doses of a medicament. The medicament carrier 14 can include various types of medicament suitable for oral or nasal inhalation. In some embodiments, the medicament can include an active pharmaceutical ingredient and an excipient. The inhalation device 12 includes a mechanism, such as, for example, a lever (not shown) to advance the medicament carrier 14 to a next dose. The inhalation device 12 includes a counter to indicate the number of doses remaining in the medicament carrier 14, as shown in FIG. 2. A user advances the medicament carrier 14 to the next dose. The user is signaled, such as, for example, by an audible click or tactile signal that the medicament carrier 14 is disposed in a ready position. The dose is administered, such as, for example, by inhalation. The medicament carrier 14 is configured to protect and/or retain the medicament disposed therein.

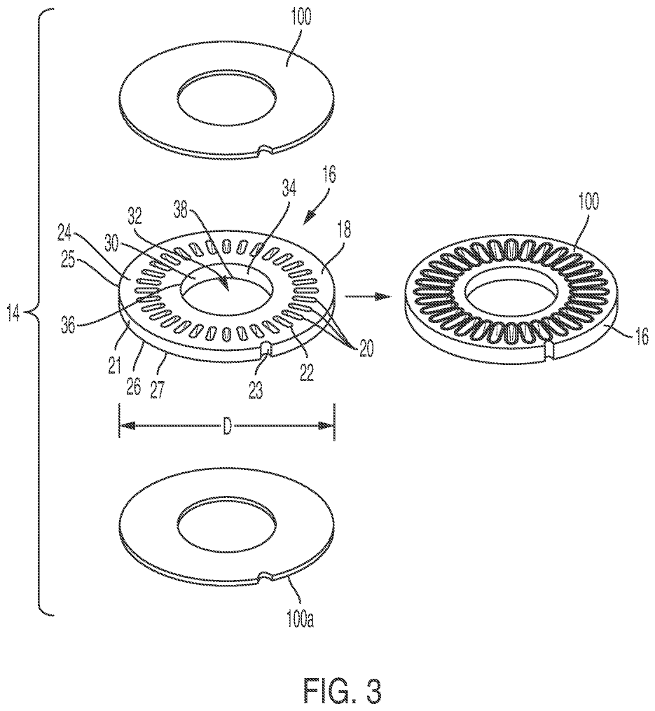

The medicament carrier 14 includes a disc 16 having a circular configuration. In some embodiments, the disc 16 may have alternate configurations, such as, for example, oval, oblong triangular, square, polygonal, irregular, uniform, non-uniform, and/or tapered. The disc 16 includes an outer diameter D and a surface 18 that defines a plurality of cavities 20. The cavities 20 are spaced apart circumferentially about the disc 16. The cavities 20 are configured for disposal of a plurality of pockets 22 such that the pockets 22 are spaced apart circumferentially about the disc 16, as shown in FIG. 3. The pockets 22 are configured to be filled with a medicament for dispensing by the inhalation device 12, as shown in FIG. 2 and described herein.

The disc 16 includes a surface 24 and a surface 26, which are planar and extend relatively parallel to each other. In some embodiments, the surfaces 24, 26 may extend at alternate orientations relative to each other, such as, for example, arcuate, transverse, perpendicular, and/or other angular orientations such as acute or obtuse or coaxial, and/or may be offset or staggered. In some embodiments, the cavities 20 can extend partially between or entirely through the surfaces 24, 26. The surfaces 24, 26 are connected by a lateral wall 21. The wall 21 is circumferentially disposed about the disc 16 at outer diameter D. The wall 21 includes a notch 23 disposed with an outer circumferential surface of the disc 16. In some embodiments, the notch 23 is configured to facilitate connection of the disc 16 with the medicament carrier 14. In some embodiments, the notch 23 is configured to facilitate guidance and/or alignment of disc 16 with a cutting block 50 of a manufacturing apparatus 40, as described herein. In some embodiments, the disc 16 can include one or a plurality of notches 23.

The surface 24 is connected with the wall 21 at an edge 25 having a blunt surface configuration. In some embodiments, the edge 25 is chamfered with a 0.4 mm cut forming a 45-degree angle along the edge 25. The edge 25 is configured to cut, slice, and/or shear a layer of foil into a shape, profile, and/or perimeter of the disc 16, as described herein. The surface 26 is connected with the wall 21 at an edge 27 having a blunt surface configuration. In some embodiments, the edge 27 is chamfered with a 0.4 mm cut forming a 45-degree angle along the edge 27. The edge 27 is configured to cut, slice, and/or shear a layer of foil into a shape, profile, and/or perimeter of the disc 16, as described herein. In some embodiments, the disc 16 includes the edge 25 and/or the edge 27 and comprises a cutting punch and/or die configuration of the manufacturing apparatus 40 employed with the cutting block 50 to cut, slice, and/or shear a layer of foil into a shape, profile, and/or perimeter of an outer diameter of the disc 16.

The disc 16 includes a surface 30 that defines an opening 32 having a circular configuration. The opening 32 defines an inner diameter of the disc 16. The surface 30 defines a lateral wall 34. The wall 34 is circumferentially disposed about the opening 32 at the inner diameter of disc 16. The surface 24 is connected with the wall 34 at an edge 36 having a blunt surface configuration. In some embodiments, the edge 36 is chamfered with a 0.4 mm cut forming a 45-degree angle along the edge 36. The edge 36 is configured to cut, slice, and/or shear a layer of foil into a shape and/or profile of the opening 32, as described herein. The surface 26 is connected with the wall 34 at an edge 38 having a blunt surface configuration. In some embodiments, the edge 38 is chamfered with a 0.4 mm cut forming a 45-degree angle along the edge 38. The edge 38 is configured to cut, slice, and/or shear a layer of foil into a shape and/or profile of the opening 32, as described herein. In some embodiments, the disc 16 includes an edge 36 and/or edge 38 and comprises a cutting punch and/or die configuration of the manufacturing apparatus 40 employed with the cutting block 50 to cut, slice, and/or shear a layer of foil into a shape and/or profile of the opening 32 and/or an inner diameter of the disc 16.

The surface 30 is engageable with a portion of the inhalation device 12 for disposal therewith. In some embodiments, the opening 32 facilitates rotation of the medicament carrier 14 relative to the inhalation device 12 for dosing and/or delivery of a medicament by oral and/or nasal inhalation. In some embodiments, the opening 32 may have alternate configurations, such as, for example, oval, oblong triangular, square, polygonal, irregular, uniform, non-uniform, and/or tapered. In some embodiments, the opening 32 is configured to facilitate guidance and/or alignment of the disc 16 with the cutting block 50, as described herein.

The disc 16 includes one or more material layers and/or membranes, as described herein, connected with one or more surfaces of the disc 16 to protect, support, and/or contain medicament, doses of medicament, and/or the pockets 22 within the disc 16, for example, within the cavities 20. In some embodiments, the disc 16 includes a flexible layer of material comprising a layer of foil 100. In some embodiments, the disc 16 includes a material layer comprising a metal foil, such as, for example, an aluminum foil or aluminum foil laminate. In some embodiments, the disc 16 includes a material layer including plastic, plastic laminates, plastic/paper laminates, or plastic/metal foil laminates.

The pockets 22 are sealed by the foil 100 to provide a medicament carrier 14 having a unit-dose package configuration. The foil 100 is configured to provide a barrier along surface 24 to protect and/or retain the medicament disposed within the pockets 22. The foil 100 is applied to the surface 24 and cut to a shape, profile, and/or perimeter of the disc 16 to form the medicament carrier 14, as shown in FIG. 3 and described herein. For example, the foil 100 is disposed across the surface 24 and adhered thereto for sealing the pockets 22 and the medicament contained therein. In some embodiments, the foil 100 is applied to the surface 24 by cold sealing, heat sealing, static sealing, and/or adhesives.

In some embodiments, the disc 16 includes a top layer of foil 100 disposed with the surface 24 and a bottom layer of foil 100a disposed with the surface 26, as shown in FIG. 3. In some embodiments, the top layer of foil 100 and/or the bottom layer of foil 100a may be provided with one or more lines of weakness and/or pierce, puncture, or perforation configurations to facilitate release of one or more doses of medicament from the medicament carrier 14 of the inhalation device 12.

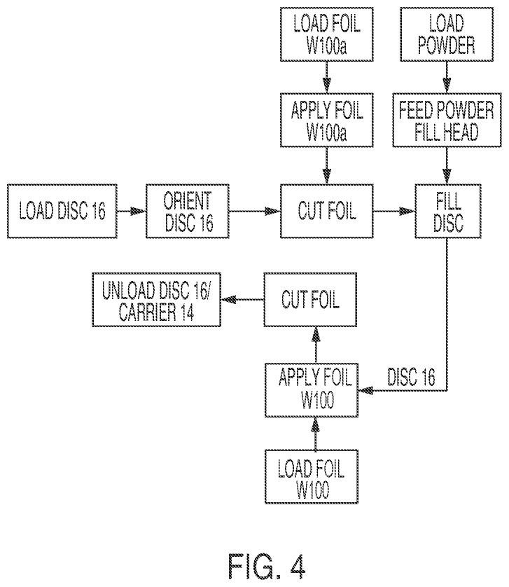

In some embodiments, the components of the manufacturing system 10 are employed to manufacture one or more components of the inhalation device 12. In one embodiment, the manufacturing system 10 includes process steps and one or more manufacturing apparatus 40, 340 and filling machines 200 employed to manufacture the medicament carrier 14 of inhalation device 12, as shown for example in FIGS. 4-10. In one embodiment, the components of the manufacturing system 10 applies one or more material layers and/or membranes, as described herein, with one or more surfaces of the disc 16 to protect, support, and/or contain medicament, doses of medicament, and/or the pockets 22 within the disc 16. The manufacturing apparatus 40 includes a cutting block 50 that cuts the material layer and/or membrane to a shape, profile, and/or perimeter of the disc 16 in connection with fabrication of the medicament carrier 14 that is assembled with the inhalation device 12, which is configured for dosing and/or delivery of a medicament by oral and/or nasal inhalation, as described herein.

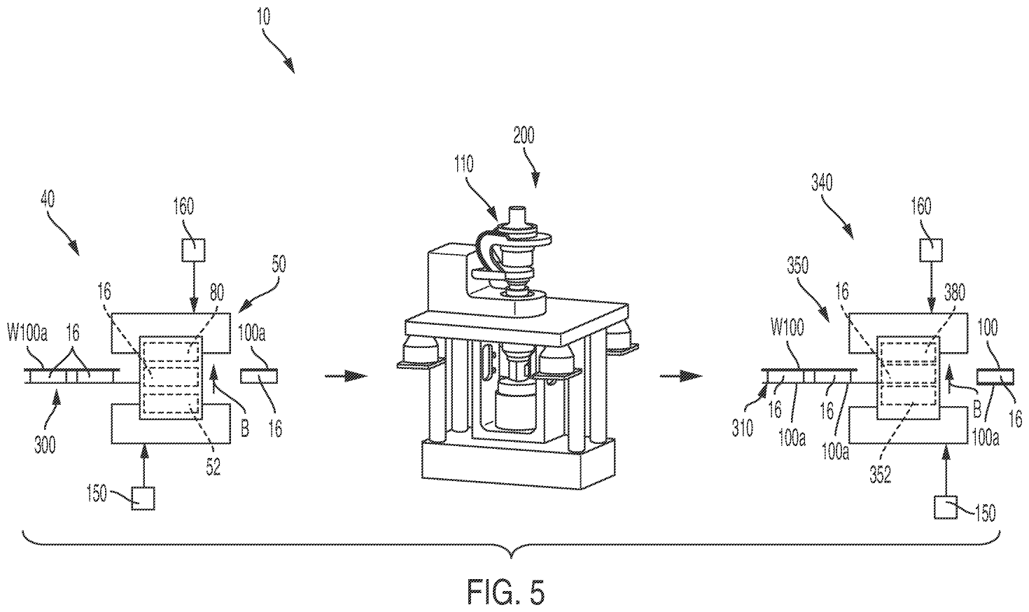

For example, as shown in FIGS. 4 and 5, the manufacturing system 10 is employed to manufacture the medicament carrier 14 of the inhalation device 12 such that the disc 16 is loaded with the manufacturing apparatus 40 including the cutting block 50. In some embodiments, the disc 16 is disposed on a conveyor belt 300 for conveying into the manufacturing apparatus 40 for the cutting of the foils 100, 100a, as described herein. In some embodiments, the disc 16 is manually loaded with the manufacturing apparatus 40.

A web of foil W100a is applied along the surface 26 of the disc 16, as shown in FIG. 5. The foil web W100a is adhesively disposed about the edge 27 of the disc 16. In some embodiments, the foil web W100a is attached, adhered, and/or sealed with the surface 26 and/or seals the cavities 20 and/or the pockets 22 to protect and/or retain medicament disposed therein. In some embodiments, the foil web W100a is applied to the surface 26 manually, via machine processing, or robotically. The disc 16, with the foil web W100a applied thereto, is manually loaded and/or conveyed into the manufacturing apparatus 40 for disposal with the cutting block 50 to cut foil web W100a.

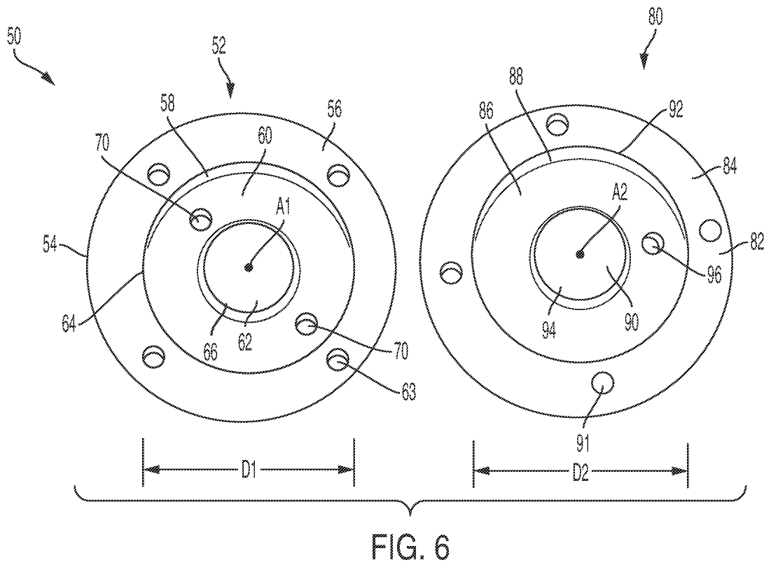

The cutting block 50 includes a member, such as, for example, a base cutting plate 52. The cutting plate 52 includes a body 54 that defines an axis A1. The body 54 includes a wall 56. The body 54 includes a surface 58 that defines a cavity 60. The wall 56 is circumferentially disposed about the cavity 60, as shown in FIG. 6, which includes a circular configuration for disposal of the disc 16. The cavity 60 defines a diameter D1 sized to accommodate disposal and sliding translation of the disc 16 within the cutting plate 52 and along axis A1. In some embodiments, the outer diameter D of the disc 16 is approximately 63 mm. In some embodiments, the diameter D1 of the cavity 60 includes a tolerance of +/−0.1 mm relative to the diameter D of the disc 16 to facilitate translation of the disc 16, as described herein. In some embodiments, the cavity 60 may have alternate configurations, such as, for example, oval, oblong triangular, square, polygonal, irregular, uniform, non-uniform, offset, staggered, and/or tapered.

The body 54 includes a guide 62 having a circular configuration for disposal within the opening 32 and a mating engagement with the surface 30 for guiding the disc 16 into position with the cutting plate 52. In some embodiments, the body 54 includes a pin (not shown) configured for mating engagement with the notch 23 to facilitate alignment and guiding of the cutting plate 52 with the disc 16. In some embodiments, the guide 62 is centrally disposed with the body 54 to align the disc 16 along axis A1. In some embodiments, the guide 62 may be disposed at alternate orientations relative to the body 54, such as, for example, angled, offset, and/or staggered.

The wall 56 includes an edge 64 such that the cutting plate 52 comprises a press configuration to cut, slice, and/or shear the foil web W100a into a shape, profile, and/or perimeter of an outer diameter of the disc 16. The guide 62 includes an edge 66 such that the cutting plate 52 comprises a press configuration to cut, slice, and/or shear the foil web W100a into a shape and/or profile of the opening 32 and/or an inner diameter of the disc 16.

The cutting block 50 includes a member, such as, for example, a lid cutting plate 80. The cutting plate 80 includes a body 82 that defines an axis A2. The body 82 includes a wall 84. The body 82 includes a surface 86 that defines a cavity 88. The wall 84 is circumferentially disposed about the cavity 88, which includes a circular configuration for disposal of disc 16. The cavity 88 includes a diameter D2 sized to accommodate disposal and sliding translation of the disc 16 within the cutting plate 80 and along axis A2. In some embodiments, the diameter D2 of the cavity 88 includes a tolerance of +/−0.1 mm relative to the diameter D of the disk 16 to facilitate translation of the disc 16, as described herein. In some embodiments, the cavity 88 may have alternate configurations, such as, for example, oval, oblong triangular, square, polygonal, irregular, uniform, non-uniform, offset, staggered, and/or tapered.

The body 82 includes a guide 90 having a circular configuration for disposal within the opening 32 and mating engagement with the surface 30 for guiding the disc 16 into position with the cutting plate 80. In some embodiments, the body 82 includes a pin (not shown) configured for mating engagement with the notch 23 to facilitate alignment and guiding of the cutting plate 80 with the disc 16. In some embodiments, the guide 90 is centrally disposed with the body 82 to align the disc 16 along axis A2. In some embodiments, the guide 90 may be disposed at alternate orientations relative to the body 82, such as, for example, angled, offset, and/or staggered.

The wall 84 includes an edge 92 such that the cutting plate 80 comprises a press configuration to cut, slice, and/or shear a layer of foil into a shape, profile, and/or perimeter of an outer diameter of the disc 16. The guide 90 includes an edge 94 such that the cutting plate 80 comprises a press configuration to cut, slice, and/or shear a layer of foil into a shape and/or profile of the opening 32 and/or an inner diameter of the disc 16.

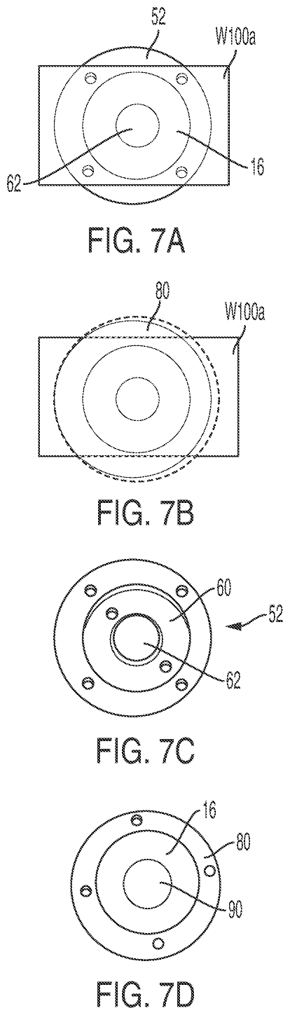

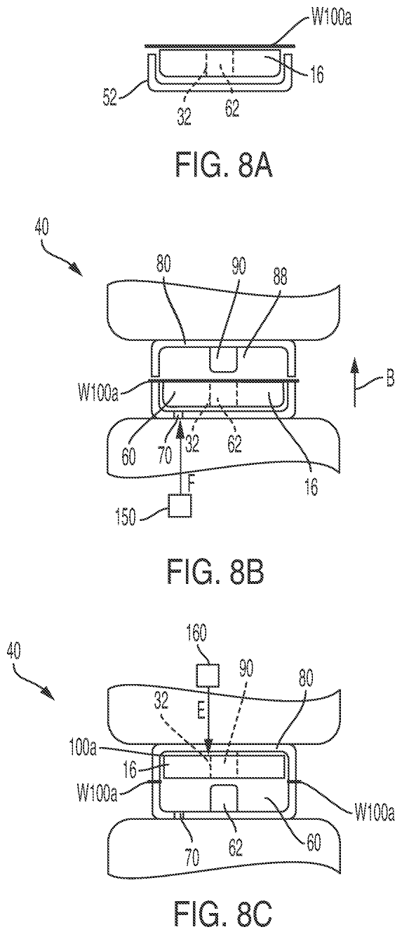

The foil web W100a with one or more of the discs 16 connected thereto is conveyed, manipulated, and/or fed into the cutting block 50 such that the opening 32 is aligned with the guide 62 and the disc 16 is disposed with the cutting plate 52, such as within the cavity 60, as shown in FIGS. 7A and 8A. Axes A1, A2 are aligned and the cutting plate 80 is translated into engagement with the cutting plate 52, as shown in FIGS. 7B and 8B, to firmly and fixedly support the foil web W100a therebetween. The foil web W100a is clamped between the cutting plates 52, 80, for example, by pins, bolts or screws disposed within the cavities 63, 91 of the cutting plates 52, 80. The dashed portion of W100a in FIG. 7B illustrates the portion of foil web W100a that is clamped between the cutting plates 52, 80 and would therefore not be visible.

The surface 58 of the cutting plate 52 includes spaced-apart openings 70 disposed circumferentially about the axis A1. The openings 70 are configured for connection with an actuator source 150 that delivers an actuator, such as, for example, a fluid including gas or liquids, air, push-rod, and/or a pin, through the openings 70. The actuator applies a force to the disc 16, as shown by arrow F in FIG. 8B, for engagement with a surface of the disc 16 to slidably translate the disc 16 from the cutting plate 52 to the cutting plate 80 for cutting the foil web W100a to a shape, profile, inner and outer diameter, and/or perimeter of the disc 16. In some embodiments, the actuator source 150 may include various mechanisms connected with the cutting block 50, such as, for example, hydraulic, pneumatic, punch, manual, spring loaded and/or gravity induced.

For example, a pneumatic air source may be attached with the cutting block 50 for delivering air through the openings 70. The air is forced through the openings 70 to engage the surface 26 to drive the disc 16 from the cutting plate 52 to the cutting plate 80 in a direction shown by arrow B in FIGS. 5 and 8B. The disc 16 translates along the guides 62, 90 from the cavity 60 into the cavity 88. FIG. 7D shows the disc 16 disposed in cavity 88 of cutting plate 80. The disc 16 translates such that the edge 27 engages the surface of foil web W100a against the edge 92 to cut, slice, and/or shear foil web W100a into the shape and profile of an outer diameter of the disc 16. The disc 16 also translates such that the edge 38 engages the surface of the foil web W100a against the edge 94 to cut, slice, and/or shear the foil web W100a into the shape and profile of an inner diameter of the disc 16.

The cutting plate 80 is disengaged from the cutting plate 52, for example, by releasing pins, bolts, or screws disposed within the cavities 63, 91. The surface 86 includes an opening 96 disposed circumferentially about the axis A2. The opening 96 is configured for connection with a release actuator source 160 that delivers an actuator, such as, for example, a fluid including gas or liquids, air, push-rod, and/or a pin, through the opening 96. In some embodiments, the release actuator may include various mechanisms, such as, for example, a fluid, hydraulic, air, and/or a pin. The release actuator applies a force to the disc 16, as shown by arrow E in FIG. 8C, for engagement with a surface of the disc 16 to remove and/or expel the disc 16 from the cutting plate 80. In some embodiments, the release actuator source 160 may include various mechanisms connected with the cutting block 50, such as, for example, hydraulic, pneumatic, punch, manual, spring loaded, and/or gravity induced. For example, a push rod (not shown) is attached with the cutting block 50 for translating the rod through the opening 96 to engage the surface 24 to remove and/or expel the disc 16 from the cutting plate 80.

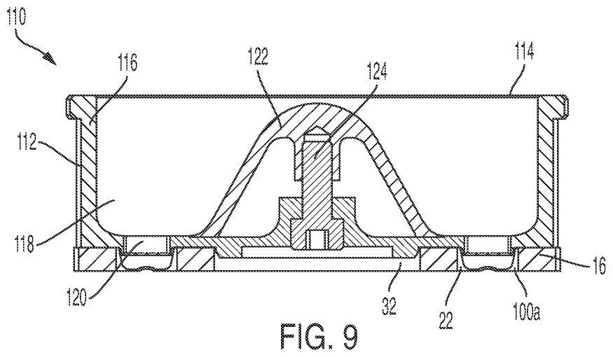

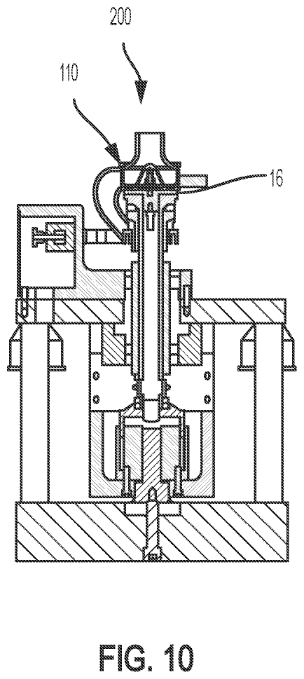

The disc 16 having the bottom foil 100a is manipulated such that the openings of the pockets 22 are oriented to receive medicament from the filling machine 200, as shown in FIGS. 5, 9, and 10. The disc 16 with the foil 100a attached thereto is conveyed, manipulated, and/or fed into the filling machine 200, as represented by the bold arrow in FIG. 5. The pockets 22 are filled with a medicament by a filling head 110, as shown in FIG. 9. The arrangement of the filling head 110 in the filling machine 200 may be as shown in FIG. 10. The filling head 110 is configured to fill the pockets 22 with a defined amount of medicament. In some embodiments, the filling head 110 is configured to layer the medicament in a linear or a non-linear configuration. In some embodiments, the filling head 110 is configured to meter the medicament into a defined volume at a consistent density, to achieve weight control and uniformity of the powder.

The filling head 110 comprises a receptacle 112. The receptacle 112 includes an inner surface 114 configured to receive the medicament. The receptacle 112 contains the medicament and provides a continuous supply of the medicament to enable filling of the pockets 22. The surface 114 defines a circumferential wall 116 and a base 118. The base 118 defines a plurality of openings 120. The openings 120 are configured for dispensing medicament into the pockets 22. The openings 120 are circumferentially disposed about a conical-shaped projection 122. The conical-shaped projection 122 is configured for fixed engagement substantially in a center of the base 118 via a fixation element, such as a screw 124. The disc 16 is removed from the filling head 110.

In some embodiments, the filling head 110 may provide a supply of one or more medicaments including, for example, powder active pharmaceutical ingredients (APIs), powder excipients, and/or any other powder in micronized form. The powder should have the particle size and flowability to be administered by inhalation (e.g., oral or nasal inhalation). The API can be any API suitable for inhalation, including an anticholinergic agent, a bronchodilator, and/or corticosteroids. The anticholinergic agents include, but are not limited to, trihexyphenidyl, benztropine mesylate, ipratropium, tiotropium, orphenadrine, atropine, flavoxate, oxybutynin, scopolamine, hyoscyamine, tolterodine, belladonna alkaloids, fesoterodine, solifenacin, darifenacin, propantheline, 5-[3-(3-Hydroxyphenoxy)azetidin-1-yl]-5-methyl-2,2-diphenylhexanamide, or a combination thereof. The bronchodilators include, but are not limited to, adrenergic bronchodilators including, but not limited to, levalbuterol, metaproterenol, pirbuterol, formoterol, terbutaline, or albuterol; anticholinergic bronchodilators including, but not limited to, aclidinium systemic, ipratropium systemic, tiotropium systemic, orumeclidinium systemic; bronchodilator combinations including, but not limited to, umeclidinium/vilanterol systemic, budesonide/formoterol systemic, fluticasone/salmeterol systemic, albuterol/ipratropium systemic, fluticasone/vilanterol systemic, olodaterol/tiotropium systemic, formoterol/mometasone systemic, formoterol/glycopyrrolate systemic, orglycopyrrolate/indacaterol systemic; and methylxanthines including, but not limited to, theophylline systemic, aminophylline systemic, or dyphylline systemic. The corticosteroids include, but are not limited to, beclomethasone, budesonide, flunisolide, fluticasone, mometasone, ciclesonide, or tiotropium. The powder may also include substances traditionally regarded as actives, drugs, and bioactive agents, as well as biopharmaceuticals (e.g., peptides, hormones, nucleic acids, gene constructs, etc.) typically employed to treat a number of conditions which is defined broadly to encompass diseases, disorders, infections, and the like. Exemplary APIs, without limitation, include antibiotics, antivirals, H2-receptor antagonists, 5HT1 agonists, 5HT3 antagonists, COX2-inhibitors, APIs used in treating psychiatric conditions such as depression, anxiety, bipolar condition, tranquilizers, APIs used in treating metabolic conditions, anticancer APIs, APIs used in treating neurological conditions such as epilepsy and Parkinson's Disease, respiratory conditions, such as for example, asthma, Chronic Obstructive Pulmonary Disease (COPD), bronchitis, bronchiolitis, emphysema, or the like. Also included are APIs used in treating cardiovascular conditions, non-steroidal anti-inflammatory APIs, APIs used in treating Central Nervous System conditions, and APIs employed in treating hepatitis.

In some embodiments, the manufacturing system 10 includes the disc 16, having the bottom foil 100a applied as described herein, being disposed on a conveyor belt 310 for handling into a manufacturing apparatus 340 for cutting of the foil 100, similar to that described herein, to apply a top material layer or membrane to the disc 16 in connection with fabrication of the medicament carrier 14. FIG. 5 represents one embodiment wherein disc 16 is transferred from filling machine 200 into manufacturing apparatus 340, with the transfer represented by the bold arrow in FIG. 5. In this embodiment, the disc 16 has been flipped over so that the foil 100a is on the bottom of disc 16. In some embodiments, the manufacturing apparatus 340 is of the same or substantially similar design to the manufacturing apparatus 40 described herein with reference to FIGS. 5-8C. In some embodiments, the manufacturing apparatus 340 can be same apparatus 40 that was used to apply the bottom foil 100a. In some embodiments, the disc 16 is manually loaded into the manufacturing apparatus 340. In some embodiments, the manufacturing system 10 includes applying a material layer or membrane to only a single surface, for example, only the surface 24 or only the surface 26, of the disc 16 in connection with fabrication of the medicament carrier 14.

A web of foil W100 is applied along the surface 24 of the disc 16, as shown in FIG. 5. The foil web W100 is adhesively disposed about the edge 25 of the disc 16. In some embodiments, the foil web W100 is attached, adhered, and/or sealed with the surface 24 and/or seals the cavities 20 and/or the pockets 22 to protect and/or retain the medicament disposed therein. In some embodiments, the foil web W100 is applied to the surface 24 manually, via machine processing, or robotically. The disc 16, with the foil web W100 applied thereto, is manually loaded and/or conveyed into the manufacturing apparatus 340, similar to the manufacturing apparatus 40 described herein, for disposal with the cutting block 350, similar to the cutting block 50 described herein, to cut the foil web W100.

The cutting block 350 includes a base cutting plate 352 and a lid cutting plate 380, similar to the plates 52, 80 described herein. One or more discs 16, each disc 16 having the bottom foil 100a applied as described herein, are connected with the foil web W100 and conveyed, manipulated, and/or fed into the cutting block 350. The opening 32 is aligned with a guide (not shown) of the cutting plate 352, similar to the guide 62 described herein, and the disc 16 is disposed with the cutting plate 352, similar to that described with regard to FIGS. 7A and 8A. The cutting plate 380 is translated into engagement with the cutting plate 352, similar to that described with regard to FIGS. 7B and 8B, to firmly and fixedly support the foil web W100 therebetween.

A pneumatic air source is attached with the cutting block 350 for delivering air through openings (not shown) of the cutting plate 352, similar to the openings 70 and that described with regard to FIGS. 6-8C. Air is forced through the openings of the cutting plate 352 to engage the disc 16 with the bottom foil 100a applied to the surface 26 for driving the disc 16 from the cutting plate 352 to the cutting plate 380, similar to that described with regard to FIGS. 6 and 8B. The disc 16 translates along guides of the cutting plates 352, 380 such that the edge 25 engages the surface of the foil web W100 against the edge 92 to cut, slice, and/or shear the foil web W100 into the shape and profile of an outer diameter of the disc 16. The disc 16 also translates such that the edge 38 engages the surface of the foil web W100 against the edge 94 to cut, slice, and/or shear the foil web W100 into the shape and profile of an inner diameter of the disc 16.

The cutting plate 380 is disengaged from the cutting plate 352, similar to that described with regard to FIGS. 6-8C, and a release actuator, such as one having the design of release actuator 160 discussed above, applies a force to the disc 16 to remove and/or expel the disc 16 from the cutting plate 380 having the top foil 100 applied to the surface 24 for the sealing cavities 20 and/or the pockets 22 to protect and/or retain the medicament disposed therein. The disc 16, including the foils 100, 100a, forms the medicament carrier 14, which is disposed within the inhalation device 12. The medicament carrier 14 includes individually packed doses of a medicament disposed within the pockets 22 and enclosed by the foils 100, 100a. In some embodiments, the inhalation device 12, similar to the systems and methods described herein, including the medicament carrier 14, as described herein, is employed in connection with treatment of a disease, illness, and/or ailment of a patient. The components of the inhalation device 12 facilitate user/patient compliance with a medication regimen. The medicament carrier 14 is engaged with the inhalation device 12 for dosing and/or delivery of a medicament by oral and/or nasal inhalation.

It is to be understood that the invention may assume various alternative variations and step sequences, except where expressly specified to the contrary. It is also to be understood that the specific devices and processes illustrated in the attached drawings and described in the specification are simply exemplary embodiments or aspects of the invention. Although the invention has been described in detail for the purpose of illustration based on what is currently considered to be the most practical and preferred embodiments or aspects, it is to be understood that such detail is solely for that purpose and that the invention is not limited to the disclosed embodiments or aspects, but, on the contrary, is intended to cover modifications and equivalent arrangements that are within the spirit and scope thereof. For example, it is to be understood that the present invention contemplates that to the extent possible, one or more features of any embodiment or aspect can be combined with one or more features of any other embodiment or aspect.

1. A method for fabricating an inhalation device, the method comprising the steps of: disposing a medicament carrier within a first member, the carrier being connected with a layer; engaging a second member with the first member to support the layer therebetween, the second member including a cutting surface and defining a cavity; and translating the carrier relative to the members to dispose the carrier within the cavity and cut the layer about the carrier.

2. The method according to claim 1, wherein the first member includes a guide and the step of disposing includes aligning the carrier with the guide.

3. (canceled)

4. The method according to claim 1, wherein the step of translating includes engaging the carrier such that the carrier translates along an axis of the members to cut the layer about the carrier.

5. The method according to claim 4, wherein the step of translating includes engaging the carrier with a fluid.

6. The method according to claim 1, further comprising the step of expelling the carrier from the cavity with a release actuator.

7. A method for fabricating an inhalation device, the method comprising the steps of: disposing a medicament dose carrier within a base plate, the base plate including an inner surface and a central guide that define a base cavity, the carrier including a disc being positioned onto the guide and connected with a foil web; engaging a lid plate including an inner surface and a central guide that define a lid cavity with the base plate to support the foil web therebetween, the lid plate further including a cutting surface having a blunt edge; engaging the carrier with an actuator such that the disc translates along the guide of the lid plate to cut the foil web about a perimeter of the disc; and expelling the carrier from the lid cavity with a release actuator.

8. A manufacturing apparatus comprising: a first plate that defines an axis and is configured for disposal of a medicament carrier; a second plate being aligned and engageable with the first plate to support a layer therebetween, the carrier being connected to the layer and at least one of the plates including a cutting surface; and an actuator being engageable with the carrier such that the carrier translates along the axis and cuts the layer about the carrier.

9. The manufacturing apparatus according to claim 8, wherein the cutting surface has a blunt edge.

10. The manufacturing apparatus according to claim 8, wherein each of the plates include an inner surface and a central guide that define a cavity.

11. The manufacturing apparatus according to claim 8, wherein at least one of the plates includes a central guide engageable with the carrier.

12. The manufacturing apparatus according to claim 8, wherein the first plate includes at least one opening for disposal of the actuator.

13. The manufacturing apparatus according to claim 8, wherein the actuator includes a fluid.

14. The manufacturing apparatus according to claim 8, wherein the actuator includes a pin.

15. The manufacturing apparatus according to claim 8, wherein the first plate includes at least one opening for disposal of the actuator and the second plate includes at least one opening for disposal of a release actuator that expels the carrier from the second plate.

16. The manufacturing apparatus according to claim 8, wherein the layer includes a foil web.

17. The manufacturing apparatus according to claim 8, wherein the layer includes a membrane cut to a profile of the carrier.

18. The manufacturing apparatus according to claim 8, wherein the carrier includes a disc having a plurality of spaced-apart pockets.

19. The manufacturing apparatus according to claim 8, wherein the layer is sealed to the carrier.

20. The manufacturing apparatus according to claim 8, wherein the carrier includes a disc having a plurality of spaced-apart dose pockets.

21. The manufacturing apparatus according to claim 8, further comprising a foil web sealed to a first planar surface of the carrier and the layer being sealed to a second planar surface of the carrier.
